(19) United States
(12) Patent Application Publication
(10) Pub. No.: US 2014/0364732 A1
(43) Pub. Date: Dec. 11, 2014

(54) METHOD OF DETECTING ELECTROCARDIOGRAM SIGNAL, METHOD OF DISPLAYING ELECTROCARDIOGRAM SIGNAL, AND ELECTROCARDIOGRAM SIGNAL DETECTING APPARATUS

(71) Applicant: SAMSUNG ELECTRONICS CO., LTD., Suwon-si (KR)

(72) Inventors: Hyung-joon LIM, Seoul (KR); Young-tae KIM, Seongnam-si (KR); Hwan SHIM, Yongin-si (KR)

(73) Assignee: SAMSUNG ELECTRONICS CO., LTD., Suwon-si (KR)

(21) Appl. No.: 14/300,550

(22) Filed: Jun. 10, 2014

Related U.S. Application Data
(60) Provisional application No. 61/833,060, filed on Jun. 10, 2013.

(30) Foreign Application Priority Data
Apr. 29, 2014 (KR) 10-2014-0051762

(51) Int. Cl. A61B 5/0472; A61B 8/08

(52) U.S. Cl. CPC A61B 5/0472; A61B 8/5292
USPC 600/440; 600/509

(57) ABSTRACT

A method of detecting an electrocardiogram signal is disclosed. The method includes measuring an electrocardiogram signal, determining a wave period based on an inclination of the measured electrocardiogram signal, and detecting an apex of the electrocardiogram signal with at least one of a maximum size and inclination in the wave period.

### CROSS-REFERENCE TO RELATED APPLICATIONS

This application claims priority from Korean Patent Application No. 10-2014-0051762, filed on Apr. 29, 2014 in the Korean Intellectual Property Office, and U.S. Provisional Application No. 61/833,060, filed on Jun. 10, 2013 in the U.S. Patent and Trademark Office, the disclosures of which are incorporated herein by reference in their entirety.

### BACKGROUND

1. Field

Apparatuses and methods consistent with exemplary embodiments relate to detection of an electrocardiogram signal, and more particularly, to a method of detecting an electrocardiogram signal, a method of displaying an electrocardiogram signal, and an electrocardiogram signal detecting apparatus, for detecting and displaying an accurate electrocardiogram signal.

2. Description of the Related Art

Diagnosis with a medical image (for example, ultrasonic waves, microwave tomography (MT), computed tomography (CT)) uses electrocardiography (ECG) signals for extraction of an image at a specific point in time in the human body as well as image information. For example, a method of synthesizing various computed tomography images and ultrasonic images for enhancing diagnosis accuracy and performing diagnosis uses electrocardiogram signals that are simultaneously measured with an image in order to extract images at the same time.

A waveform of an electrocardiogram signal is represented as a curve of a potential difference and current generated by heart systole. In general, in one period of the electrocardiogram signal, a P wave, a Q wave, an R wave, an S wave, and a T wave are continuously generated. A P wave refers to characteristics during atrium systole. A series of a Q wave, an R wave, and an S wave (QRS complex) refers to characteristics during ventricle systole. A T wave refers to characteristics during ventricle diastole.

Among the aforementioned waves, an R wave with the greatest amplitude has been frequently used, and various methods of detecting an R wave from electrocardiogram have been proposed. However, according to related art technologies, only amplitude of an R wave is sued, and thus, an accurate R wave may not be detected from abnormal electrocardiogram.

### SUMMARY OF THE INVENTION

Exemplary embodiments may overcome the above disadvantages and other disadvantages not described above. Also, exemplary embodiments are not required to overcome the disadvantages described above, and an exemplary embodiment may not overcome any of the problems described above.

Exemplary embodiments provide a method of detecting an electrocardiogram signal and an electrocardiogram signal detecting apparatus, for detecting an accurate R wave even in abnormal electrocardiogram.

According to an aspect of an exemplary embodiment, there is provided a method of detecting an electrocardiogram signal includes measuring an electrocardiogram signal, determining a wave period based on inclination of the measured electrocardiogram signal, and detecting an apex of the electrocardiogram signal with at least one of a maximum size and inclination in the wave period.

The determining the wave period may include determining the wave period based on a variation value for each period of the measured electrocardiogram signal and a differential value of the variation value.

The determining the wave period may include determining a maximum threshold and a minimum threshold for calculation of the wave period based on an average value and a maximum value of the differential value of the variation value, the detecting may include detecting an apex with the maximum differential value of the variation value among points with the differential value of the variation value, greater than the maximum threshold, and a start point and an end point of an electrocardiogram signal period for maintaining a state, in which the differential value of the variation value is smaller than the minimum threshold based on the apex, for a period of time, may be set as a start point and an end point of the wave period, respectively.

The determining the wave period may include setting a start point of an electrocardiogram signal period for maintaining a state, in which the differential value of the variation value is greater than a first threshold, for a period of time or more, as a start point of the wave period.

The detecting the apex may include setting a point of the electrocardiogram signal with at least one of the maximum size and inclination in the wave period, as the apex.

The method may further include displaying a waveform of the electrocardiogram signal on a display.

The wave period may be a QRS complex period.

According to an aspect of another exemplary embodiment, there is provided a method of displaying an electrocardiogram signal includes measuring an electrocardiogram signal while irradiating a body portion with an ultrasonic signal and receiving a reflected signal, configuring an ultrasonic image based on the received signal, determining an wave period based on inclination of the measured electrocardiogram signal, detecting an apex of the electrocardiogram signal with a maximum size in the wave period, determining a waveform of the electrocardiogram signal based on the wave period and the apex, and displaying the determined waveform of the electrocardiogram signal together with the configured ultrasonic image.

The displaying the determined waveform of the electrocardiogram signal together with the configured ultrasonic image may include synchronizing and displaying the waveform of the electrocardiogram signal and the ultrasonic image.

According to an aspect of another exemplary embodiment, there is provided an electrocardiogram signal detecting apparatus includes an electrocardiogram signal measurer configured to measure an electrocardiogram signal, a wave period determiner configured to determine a wave period based on inclination of the measured electrocardiogram signal, and an apex detector configured to detect an apex of the electrocardiogram signal with at least one of a maximum size and inclination in the wave period.

The wave period determiner may be configured to determine the wave period based on a variation value for each period of the received electrocardiogram signal and a differential value of the variation value.

The wave period determiner may be configured to determine a maximum threshold and a minimum threshold for calculation of the wave period based on an average value and a maximum value of the differential value of the variation value, detects an apex with the maximum differential value of the variation value among points with the differential value of the variation value, greater than the maximum threshold, and sets a start point and an end point of an electrocardiogram signal period for maintaining a state, in which the differential value of the variation value is smaller than the minimum threshold based on the apex, for a period of time, as a start point and an end point of the wave period, respectively.

The wave period determiner may be configured to set a start point of an electrocardiogram signal period for maintaining a state, in which the differential value of the variation value is greater than a first threshold, for at least a period of time, as a start point of the wave period.

The apex detector may set a point of the electrocardiogram signal with at least one of the maximum size and inclination in the wave period, as the apex.

The electrocardiogram signal detecting apparatus may further include a display configured to display a waveform of the electrocardiogram signal.

The wave period may be a QRS complex period.

### BRIEF DESCRIPTION OF THE DRAWING

The above and/or other aspects will be more apparent by describing certain exemplary embodiments with reference to the accompanying drawings, in which:

FIG. 1 is a block diagram illustrating a structure of an electrocardiogram signal detecting apparatus according to an exemplary embodiment;

FIG. 2 is a diagram illustrating an electrocardiogram signal;

FIGS. 3, 4 and 5 are flowcharts of a method of detecting an electrocardiogram signal by an electrocardiogram signal detecting apparatus 100 according to an exemplary embodiment;

FIG. 6 is a diagram illustrating waveforms of an electrocardiogram signal and a differential value of a variation value of an electrocardiogram signal according to an exemplary embodiment;

FIG. 7 is a diagram illustrating a maximum threshold and a minimum threshold together with an electrocardiogram signal waveform according to an exemplary embodiment;

FIG. 8 is a diagram illustrating a method of detecting a wave period of an electrocardiogram signal in an offline mode according to an exemplary embodiment;

FIG. 9 is a diagram illustrating a method of detecting a wave period of an electrocardiogram signal in an online mode according to an exemplary embodiment;

FIG. 10 is a diagram illustrating a method of obtaining an apex (R wave) in an online mode according to an exemplary embodiment;

FIG. 11 is a diagram detected and displayed T wave and Toffset wave;

FIG. 12 is a flowchart of a method of displaying an electrocardiogram signal according to an exemplary embodiment;

FIG. 13 is a diagram illustrating registration of electrocardiogram signals and images of a medical image displaying apparatus according to an exemplary embodiment;

FIG. 14 is a flowchart of a method of displaying an electrocardiogram signal of a medical image displaying apparatus according to another exemplary embodiment;

FIG. 15 is a diagram illustrating a case in which change in consecutive parameters is measured; and

FIGS. 16 and 17 are diagrams illustrating a case in which change in an electrocardiogram signal is measured in a predetermined period.

### DETAILED DESCRIPTION OF EXEMPLARY EMBODIMENTS

Certain exemplary embodiments will now be described in greater detail with reference to the accompanying drawings.

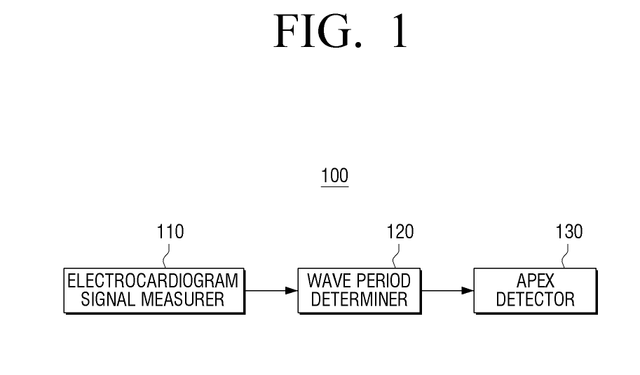

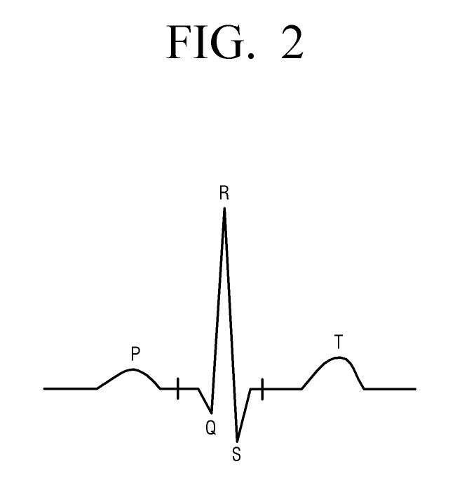

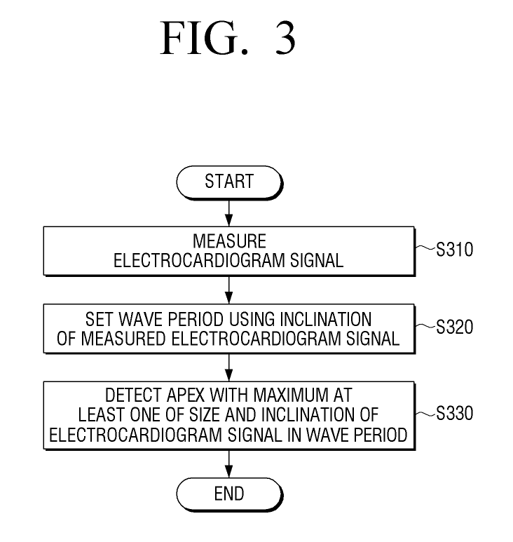

FIG. 1 is a block diagram illustrating a structure of an electrocardiogram signal detecting apparatus 100 according to an exemplary embodiment. FIG. 2 is a diagram illustrating an electrocardiogram signal. FIGS. 3, 4 and 5 are flowcharts of a method of detecting an electrocardiogram signal by the electrocardiogram signal detecting apparatus 100 according to an exemplary embodiment.

Referring to FIG. 1, the electrocardiogram signal detecting apparatus 100 according to an exemplary embodiment includes an electrocardiogram signal measurer 110, a wave period determiner 120, and an apex detector 130.

The electrocardiogram signal measurer 110 measures an electrocardiogram signal (S310, S410). In detail, when heart muscle is depolarized at each heartbeat, the electrocardiogram signal measurer 110 detects a minute electrical signal from the skin through an electrode pair attached to the skin. During a rest phase, heart muscle cells exhibit negative electric charges which are referred to as phase boundary potential. The negative electric charges are reduced in amount due to introduction of positive ions such as Na+ and Ca++ and depolarized to cause heart systole. During each heart beat, the heart has a depolarization waveform that is orderly spread over the ventricle from a signal output from the sinoatrial node. A waveform with a low voltage detected by a pair of electrodes may be represented on a display in the form of curve.

As illustrated in FIG. 2, in general, in one period of an electrocardiogram signal, a P wave, a Q wave, an R wave, an S wave, and a T wave are continuously generated.

A P wave refers to characteristics during atrium systole, a series of a Q wave, an R wave, and an S wave (QRS complex) refers to characteristics during ventricle systole, and a T wave refers to characteristics during ventricle diastole.

The wave period determiner 120 determines a wave period using an inclination (slope) of the received electrocardiogram signal (S320). Here, the wave period may be the QRS complex period. The QRS complex period refers to an electrocardiogram signal period between a start point of the QRS complex and an end point of the QRS complex based on a point in which the size (voltage and y axis) of the electrocardiogram signal is peak (greatest).

The wave period determiner 120 calculates a variation value for each period of the received electrocardiogram signal and a differential value of the variation value in order to obtain the inclination of the received electrocardiogram signal (S420 and S520).

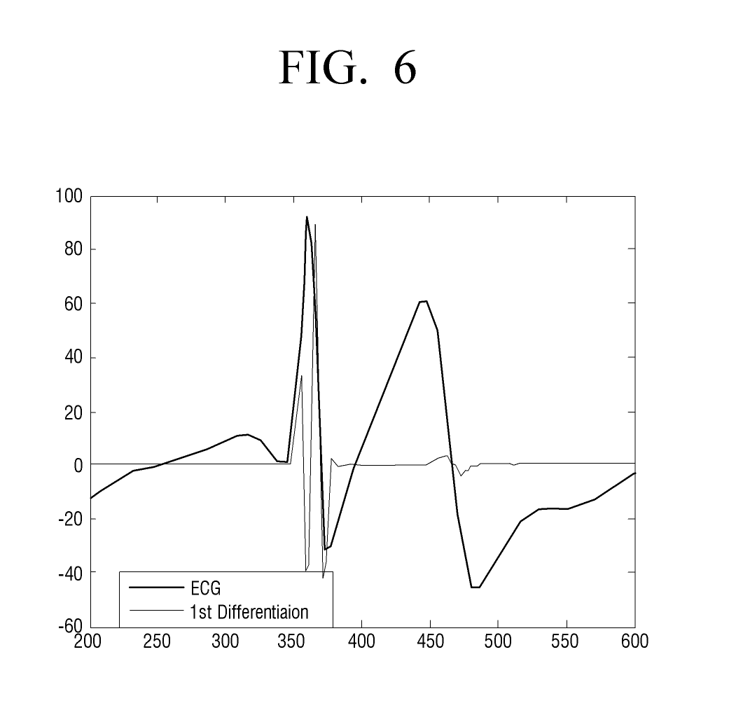

FIG. 6 is a diagram illustrating waveforms of an electrocardiogram signal and a differential value of a variation value of the electrocardiogram signal according to an exemplary embodiment.

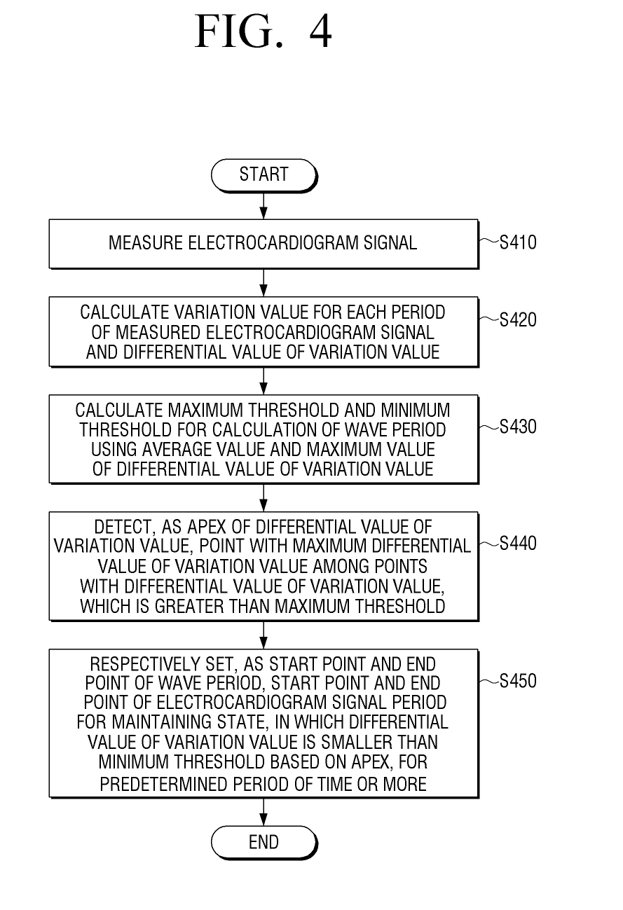

In the exemplary embodiment illustrated in FIG. 6, the electrocardiogram signal detecting apparatus 100 may calculate a variation value for each predetermined period of an electrocardiogram signal detected for a predetermined period of time and a differential value of the variation value. According to the method of detecting the electrocardiogram signal for the period of time and calculating the variation value and the differential value thereof, the electrocardiogram signal is not processed in real time, and thus, the method is defined as an offline mode. FIG. 4 is a flowchart of a method of detecting an electrocardiogram signal in an offline mode.

The wave period determiner 120 may determine the wave period using a variation value for each period of the received electrocardiogram signal and a differential value of the variation value.

The apex detector 130 detects an apex of the electrocardiogram signal with at least one of a maximum size and inclination in the wave period (S330). Here, the apex of the electrocardiogram signal with the at least one of the maximum size and inclination refers to an R peak.

As described above, when the wave period of the electrocardiogram signal and the apex are determined, an accurate waveform of the electrocardiogram signal may be determined and displayed on a display or the like.

In an offline mode, the wave period determiner 120 calculates a maximum threshold and a minimum threshold for calculation of the wave period using an average value and maximum value of the differential value of the variation value (S430). The maximum threshold may be set as a value corresponding to a predetermine ratio of the maximum value of the differential value of the variation value. Similarly, the minimum threshold may be set as a value corresponding to a predetermine ratio of the average value of the differential value of the variation value.

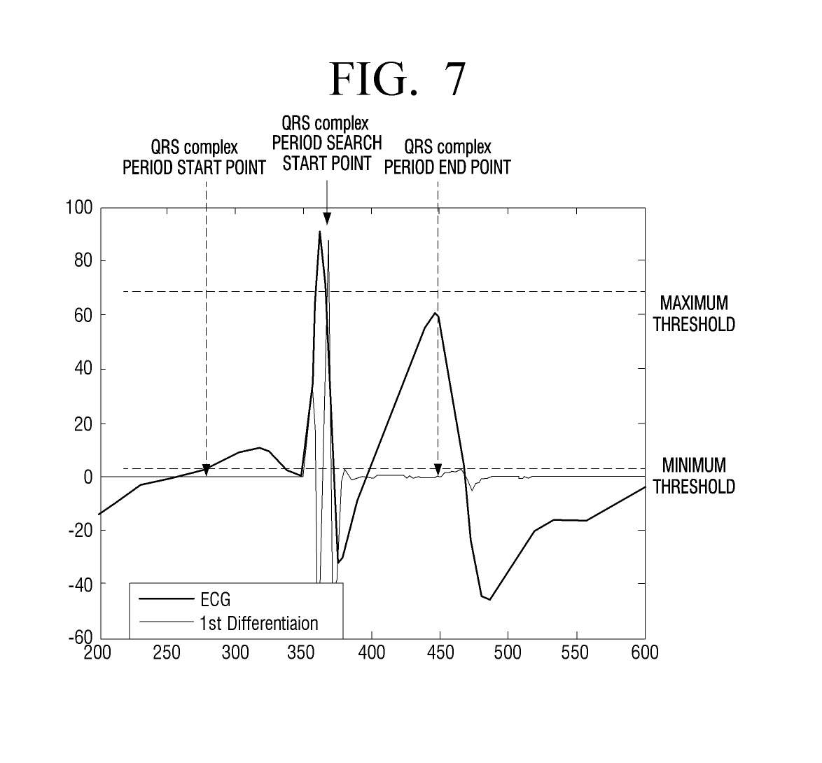

FIG. 7 is a diagram illustrating a maximum threshold and a minimum threshold together with an electrocardiogram signal waveform according to an exemplary embodiment.

The apex detector 130 may determine a point of the electrocardiogram signal with at least one of a maximum size and inclination in a wave period as an apex. As illustrated in FIG. 7, the apex may be appropriately detected from a point with greatest amplitude of an electrocardiogram signal in a QRS complex period.

In this case, the wave period determiner 120 may respectively set, as a start point and an end point of the wave period, a start point and an end point of an electrocardiogram signal period for maintaining a state, in which a differential value of the variation value is smaller than the minimum threshold based on an apex of an differential value of a variation value greater than the maximum threshold, for a predetermined period of time ‘t’ or more (S440). In detail, when a state in which a differential value of the variation value is smaller than minimum threshold for a predetermined period of time ‘t/2’ or more is maintained in a left direction from the apex of the differential value of the variation value, a start point of the electrocardiogram signal period may be set as a start point of the QRS complex period. Similarly, when a state in which a differential value of the variation value is smaller than minimum threshold for a predetermined period of time ‘t/2’ or more is maintained in a right direction from the apex of the differential value of the variation value, an end point of the electrocardiogram signal period may be set as an end point of the QRS complex period (S450).

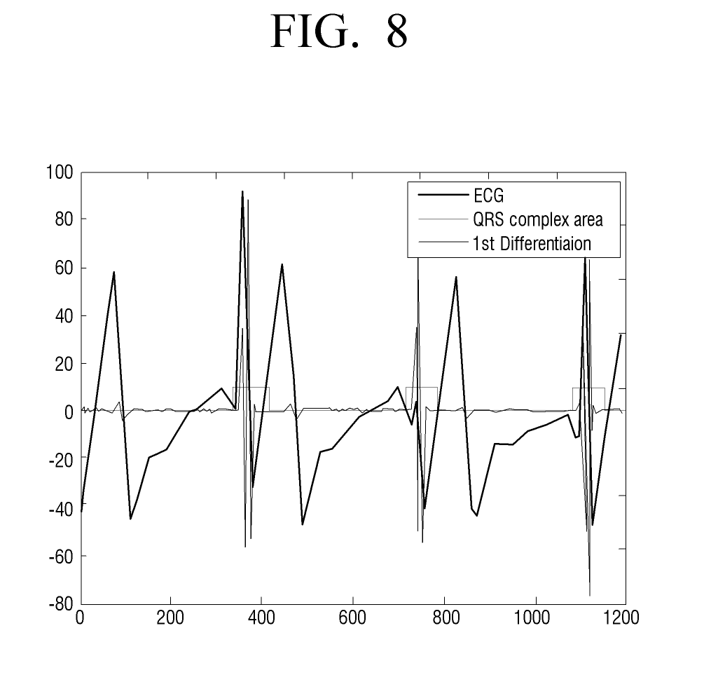

FIG. 8 is a diagram illustrating a method of detecting a wave period of an electrocardiogram signal in an offline mode.

As seen from FIG. 8, according to the aforementioned method, the wave period of the electrocardiogram signal may be detected in the offline mode.

Unlike in the aforementioned embodiment, an electrocardiogram signal may be detected and processed in real time, which is defined as an offline mode.

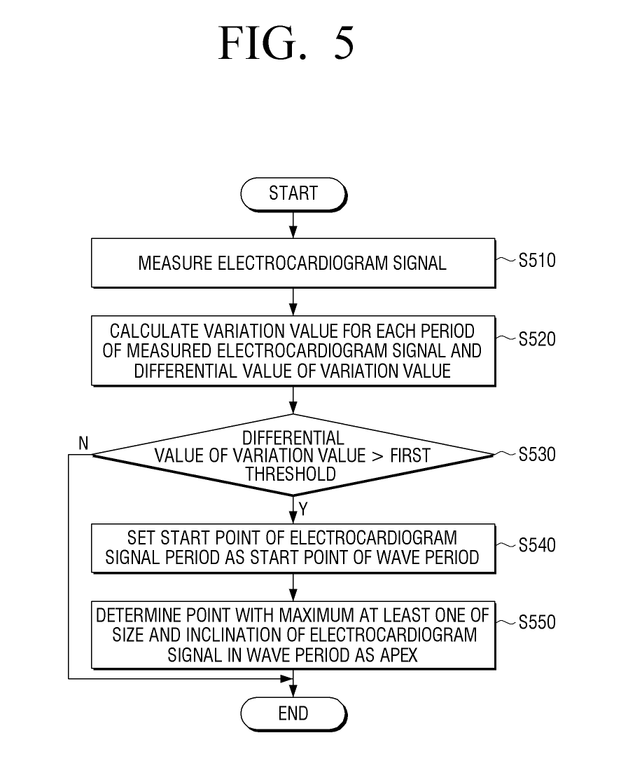

FIG. 5 is a flowchart of a method of detecting an electrocardiogram signal in an online mode. Like in the aforementioned method, the electrocardiogram signal measurer 110 detects an electrocardiogram signal (S510) and calculates a variation value for each period of the measured electrocardiogram signal and a differential value of the variation value (S520). In this case, since the current mode is an online mode, the electrocardiogram signal may be detected and the calculation may be performed for a shorter period of time than the aforementioned period of time.

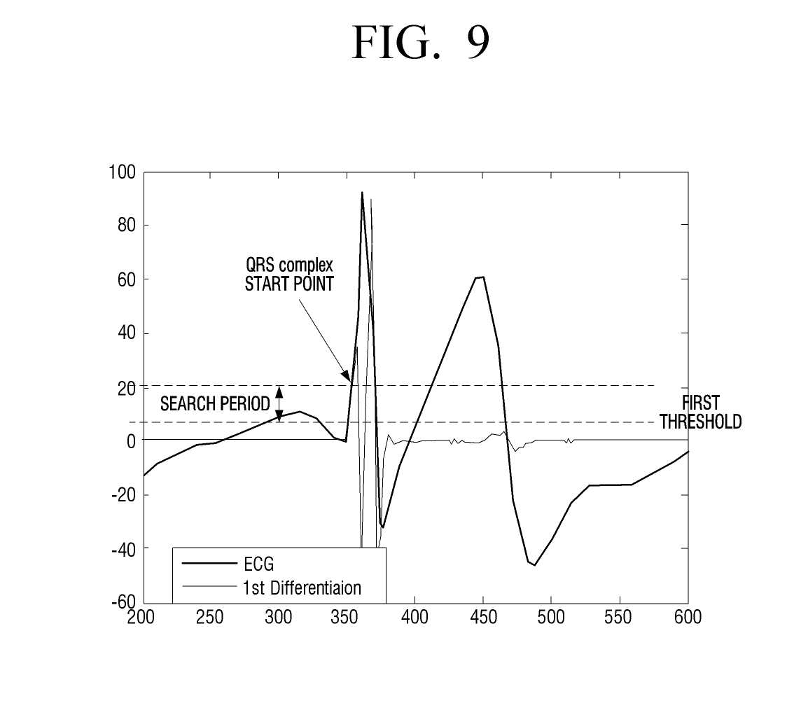

FIG. 9 is a diagram illustrating a method of detecting a wave period of an electrocardiogram signal in an online mode.

As illustrated in FIG. 9, the wave period determiner 120 may set, as a start point of the wave period, a start point of an electrocardiogram signal period for maintaining a state, in which a differential value of the variation value is greater than a predetermined first threshold, for a predetermined period of time or more (S530 and S540).

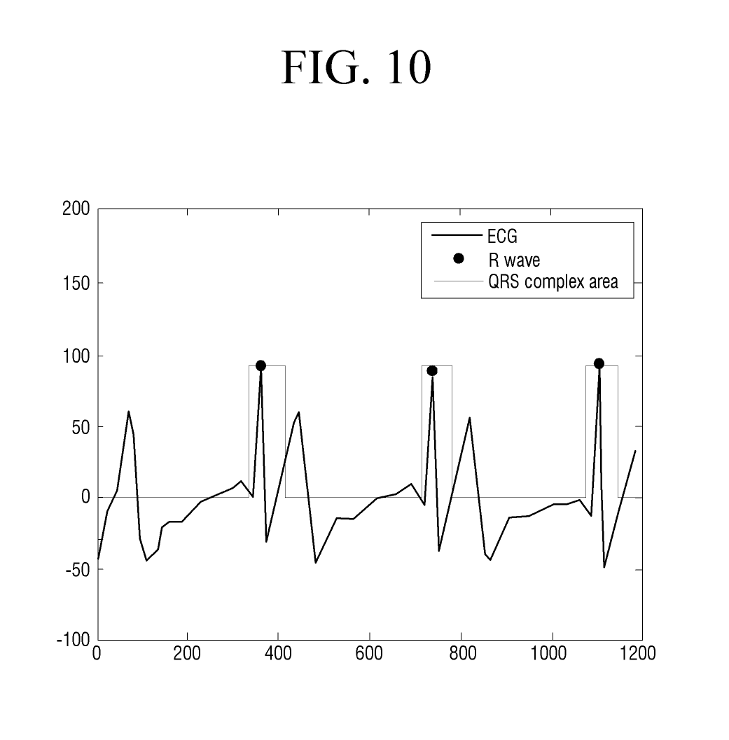

FIG. 10 is a diagram illustrating a method of obtaining an apex (R wave) in an online mode.

Referring to FIG. 10, the apex detector 130 may set a point of the electrocardiogram with at least one of the maximum size and inclination signal in the obtained wave period as an apex (S550). After the apex is detected, the aforementioned T wave may be detected or a Toffset wave may be detected.

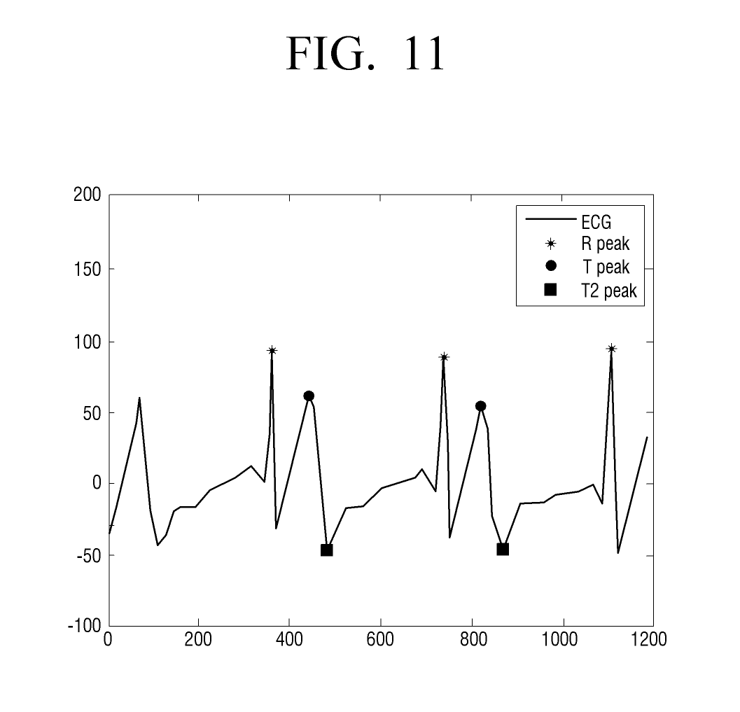

FIG. 11 is a diagram detected and displayed T wave and Toffset wave.

As illustrated in FIG. 11, after an R wave is detected, T wave or Toffset wave may be obtained based on an R wave according to diagnosis necessity or user requirements. In an online mode in which the R wave of the electrocardiogram signal is detected online, when a start point of a QRS complex period is determined, an apex with a value greater than a threshold of an electrocardiogram signal as an R wave, like in an offline mode.

When the electrocardiogram signal is detected using the aforementioned method, application is possible in various medical equipments.

The aforementioned electrocardiogram signal detecting apparatus 100 may be embodied as various medical equipments.

The various aforementioned modules of the electrocardiogram signal detecting apparatus 100 may be embodied as a controller (not shown). The controller controls an overall operation of the electrocardiogram signal detecting apparatus 100.

The controller may include a hardware configuration such as a micro processing unit (MPU), a central processing unit (CPU), a cache memory, a data bus, etc., and a software configuration such as an operating system (OS) and an application for execution of a specific purpose. A control command for each component for an operation of the electrocardiogram signal detecting apparatus 100 is read according to a system clock, and an electrical signal is generated according to the read control command to operate each component of hardware.

In addition, the electrocardiogram signal detecting apparatus 100 may further include a display (not shown) for displaying the detected electrocardiogram signal waveform.

The display may be designed as various display panels. That is, the display may be embodied using various display technologies such as an organic light emitting diode (OLED), a liquid crystal display (LCD) panel, a plasma display panel (PDP), a vacuum fluorescent display (VFD), a field emission display (FED), an electro luminescence display (ELD), etc. The display panel may be mainly a light emitting type panel, but a reflective type display (e.g., E-ink, P-ink, and photonic crystal) is not excluded. In addition, the display panel may be embodied as a flexible display, a transparent display, etc. In addition, a plurality of display panels may be used.

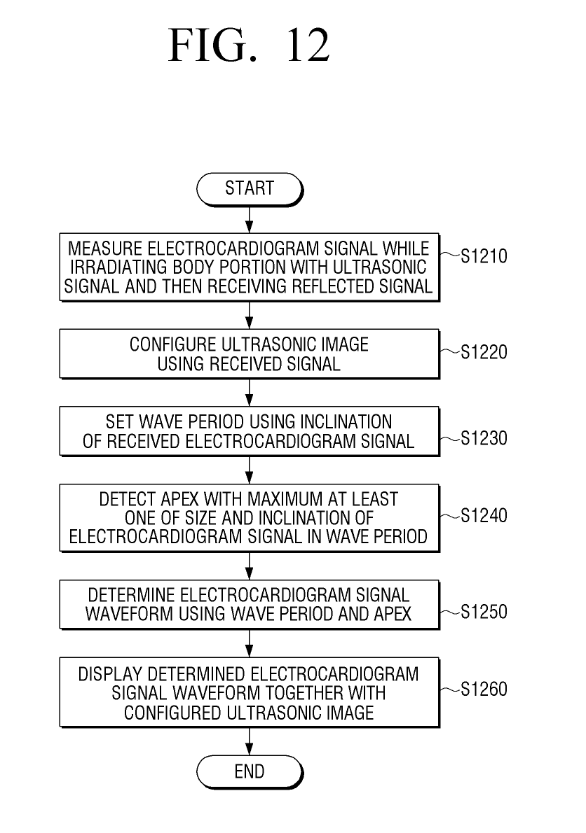

FIG. 12 is a flowchart of a method of displaying an electrocardiogram signal according to an exemplary embodiment.

A medical image displaying apparatus (not shown) according to an exemplary embodiment synchronizes and displays an ultrasonic image and an electrocardiogram signal.

To this end, first, the medical image displaying apparatus measures an electrocardiogram signal while irradiating a body portion with an ultrasonic signal and then receiving a reflected signal (S1210).

In addition, an ultrasonic image is configured using the received signal (S1220).

In this case, the ultrasonic image may be any one of a brightness (B)-mode image and a color Doppler (C)-mode image.

The B-mode image is configured by processing the body portion irradiated with ultrasonic waves with black and white using an ultrasonic echo signal reflected from the body portion. When a distance to the human body is given in a horizontal axis and amplitude of the reflected echo is given in a vertical axis, amplitude may be replaced with brightness of as dot. In this regard, the B-mode image may be configured as a black and white image in this manner.

The C-mode image is configured by processing the body portion irradiated with ultrasonic waves with colors using the ultrasonic echo signal reflected from the body portion. For example, in response to an ultrasonic echo signal being received to cause frequency deviation according to the Doppler effect, the medical image displaying apparatus may calculate the deviated frequency to measure blood flow velocity. In addition, the C-mode image may be configured using the blood flow velocity.

The medical image displaying apparatus determines a wave period using an inclination of the measured electrocardiogram signal (S1230).

In addition, the medical image displaying apparatus detects an apex with a maximum size of the electrocardiogram signal from the wave period (S1240).

Wave period setting and apex detection may be performed using the aforementioned method.

In detail, in an offline mode, the medical image displaying apparatus may calculate a variation value for each predetermined period of an electrocardiogram signal detected for a predetermined period of time and a differential value of the variation value. In addition, the medical image displaying apparatus calculates a maximum threshold and a minimum threshold for calculation of the wave period using an average value and maximum value of the differential value of the variation value. In addition, the medical image displaying apparatus may determine, as an apex of a differential value of a variation value, a point with a maximum differential value of the variation value among points with a differential value of the variation value, which is greater than the maximum threshold. In addition, the medical image displaying apparatus may respectively set, as a start point and an end point of the wave period, a start point and an end point of an electrocardiogram signal period for maintaining a state, in which a differential value of the variation value is smaller than the minimum threshold based on an apex of the differential value of the variation value, for a predetermined period of time ‘t’ or more. The medical image displaying apparatus may determine an apex of the electrocardiogram signal between the start point and the end point of the wave period.

In an online mode, the medical image displaying apparatus may calculate a variation value for each predetermined period of an electrocardiogram signal detected for a short period of time. The medical image displaying apparatus may set, as a start point of the wave period, a start point of an electrocardiogram signal period for maintaining a state, in which a differential value of the variation value is greater than a predetermined first threshold, for a predetermined period of time or more. In addition, the medical image displaying apparatus may set a point with at least one of the maximum size and inclination of the electrocardiogram signal in the obtained wave period as an apex.

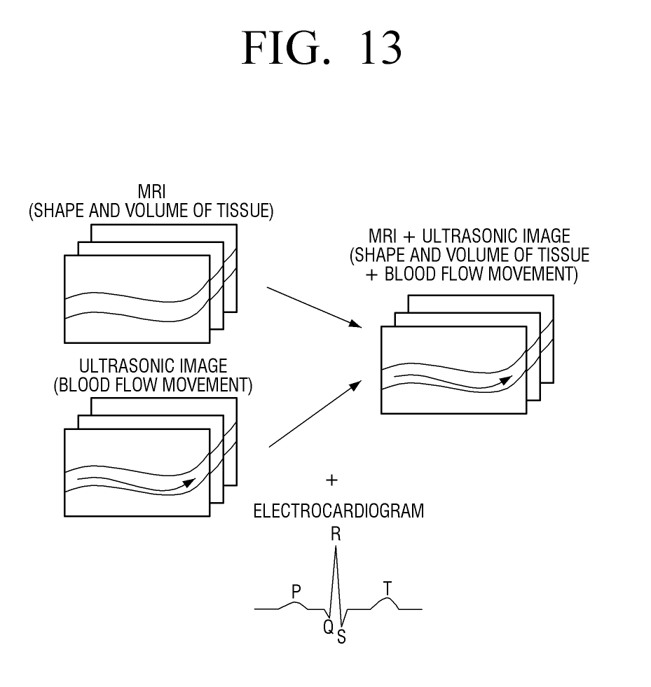

FIG. 13 is a diagram illustrating registration of electrocardiogram signals and images of a medical image displaying apparatus according to an exemplary embodiment.

The medical image displaying apparatus determines the electrocardiogram signal waveform using the wave period and the apex (S1250) and displays the determined electrocardiogram signal waveform together with the configured ultrasonic image (S1260).

For medical diagnosis, one image formed via registration of images measured by various equipments may be used to enhance the accuracy of diagnosis. Recently, a method of registering an ultrasonic image, computed tomography (CT), and microwave tomography (MT) in real time to enhance the accuracy of tumor removal in brain tumor removal surgery has been proposed. In this case, a registration point in time of images is based on a point in time in response to an R wave (apex) of electrocardiogram being generated.

The medical image displaying apparatus according to exemplary embodiments may register, synchronize, and display MRI images and ultrasonic images based on an electrocardiogram waveform, as illustrated in FIG. 13.

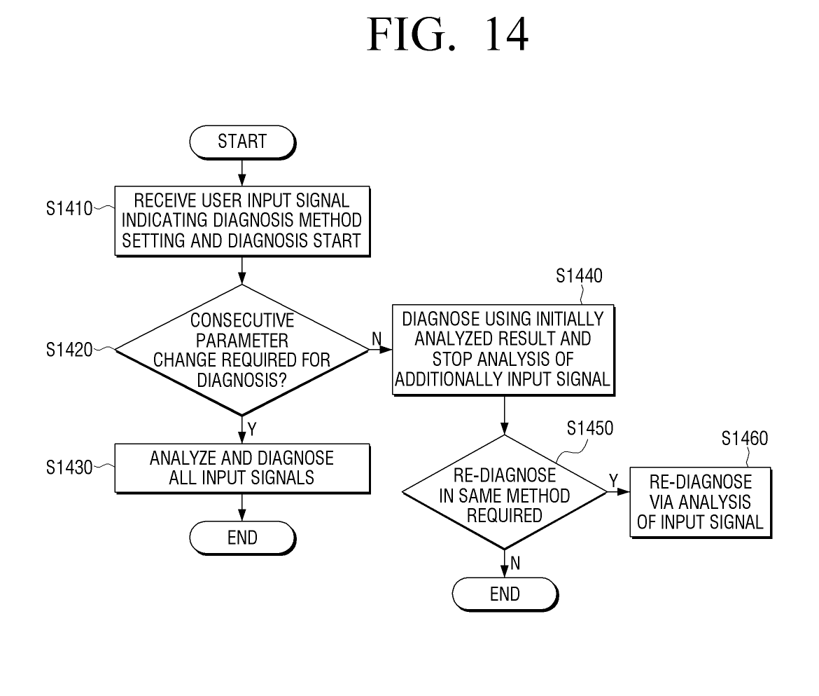

FIG. 14 is a flowchart of a method of displaying an electrocardiogram signal of a medical image displaying apparatus according to another exemplary embodiment.

Referring to FIG. 14, the medical image displaying apparatus receives a user input signal indicating diagnosis method setting input and diagnosis start (S1410).

According to a diagnosis method selected by a user, combinations of wave pairs of end of systole from end of diastole, a next R peak from an R peak, a T peak from an R peak, etc. from an electrocardiogram signal. The same method as the aforementioned method of detecting a QRS complex period may be used. An ultrasonic image frame at the same point in time is also selected.

When consecutive parameter change is required for medical diagnosis (S1420-Y), all input signals are analyzed and diagnosed (S1430).

In this case, when the diagnosis method selected by the user requires consecutive value change (online mode) (S1420-Y), a required wave is detected via the aforementioned process and required parameters are calculated and displayed on a screen whenever an electrocardiogram signal and an ultrasonic signal are received.

However, when a wave pair is detected once and then diagnosis is performed in the period (offline mode), a required electrocardiogram signal is detected and then the detection is stopped (S1440) and images required for diagnosis are displayed on a screen. Then when it is determined that the user needs to be re-measured (S1450-Y), a signal indicating start is input to the medical image displaying apparatus (S1460).

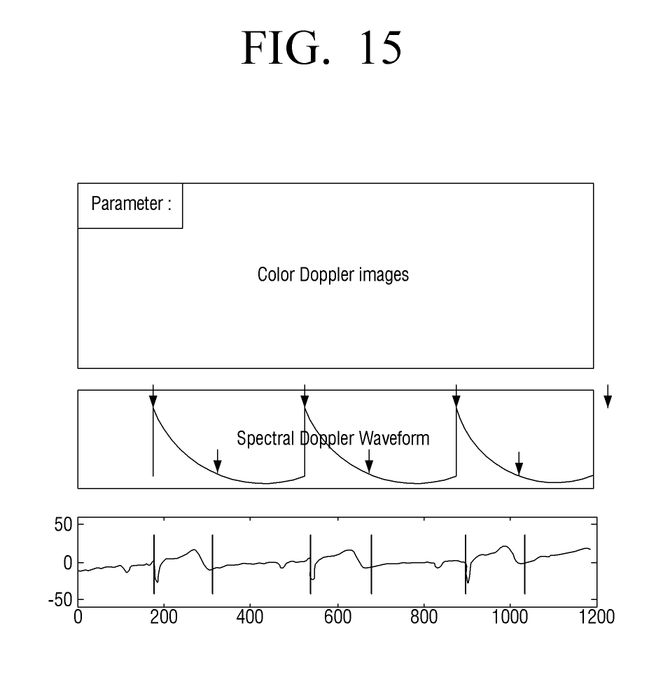

FIG. 15 is a diagram illustrating a case in which change in consecutive parameters is measured.

FIG. 15 illustrates the case in which a Doppler mode of ultrasonic waves and an electrocardiogram signal are used. In this case, the electrocardiogram signal is detected in real time and is displayed together with an ultrasonic image, and parameters required for diagnosis are displayed on an image. Then whether an electrocardiogram signal is newly detected, the aforementioned process is repeated.

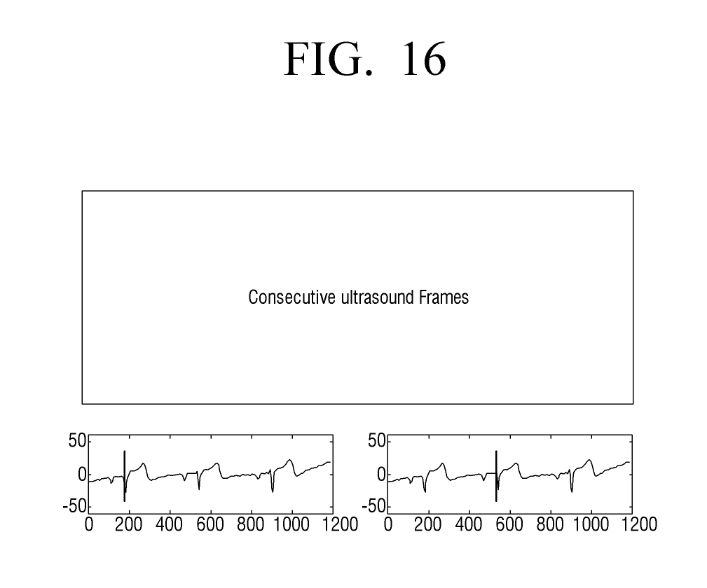

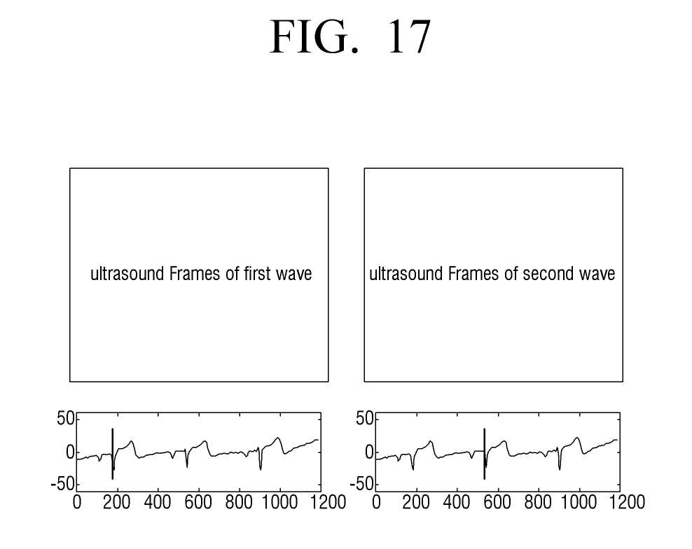

FIGS. 16 and 17 are diagrams illustrating a case in which change in an electrocardiogram signal is measured in a predetermined period.

FIG. 16 illustrates an example in which diagnosis is performed using consecutive images in an electrocardiogram signal period. FIG. 17 illustrates an example in which two ultrasonic image frames respectively corresponding to two electrocardiogram signal periods are used for diagnosis.

Although the aforementioned exemplary embodiments have been described in terms of an ultrasonic image, the inventive concept may also be applied to a medical image displaying apparatus such as an X-ray, a CT, an MRI, etc. in the same way.

The aforementioned method of measuring an electrocardiogram signal may be stored in a non-transitory computer readable medium for recording thereon a program. Here, the non-transitory computer readable medium is a medium that semi-permanently stores data and from which data is readable by a device, but not a medium that stores data for a short time, such as register, a cache, a memory, and the like. In detail, the aforementioned various applications or programs may be stored in the non-transitory computer readable medium, for example, a compact disc (CD), a digital versatile disc (DVD), a hard disc, a Blu-ray disc, a universal serial bus (USB), a memory card, a read only memory (ROM), and the like, and may be provided.

The aforementioned method of measuring an electrocardiogram signal may be embedded and provided in a hardware IC chip in the form of embedded software and may be included in components of the aforementioned electrocardiogram signal detecting apparatus 100 or the medical image displaying apparatus.

According to the various aforementioned exemplary embodiments, a method of detecting an electrocardiogram signal and an electrocardiogram signal detecting apparatus may detect an accurate R wave even in abnormal electrocardiogram.

The foregoing exemplary embodiments and advantages are merely exemplary and are not to be construed as limiting the inventive concept. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. A method of detecting an electrocardiogram signal, the method comprising:
measuring an electrocardiogram signal;
determining a wave period based on an inclination of the measured electrocardiogram signal; and
detecting an apex of the electrocardiogram signal with at least one of a maximum size and inclination in the wave period.

2. The method as claimed in claim 1, wherein the determining the wave period comprises determining the wave period based on a variation value for each period of the measured electrocardiogram signal and a differential value of the variation value.

3. The method as claimed in claim 2, wherein:
the determining the wave period comprises determining a maximum threshold and a minimum threshold for determining the wave period based on an average value and a maximum value of the differential value of the variation value;
the detecting the apex comprises detecting an apex with the maximum differential value of the variation value among points with the differential value of the variation value, greater than the maximum threshold; and
a start point and an end point of an electrocardiogram signal period for maintaining a state, in which the differential value of the variation value is smaller than the minimum threshold based on the apex, for a period of time, are set as a start point and an end point of the wave period, respectively.

4. The method as claimed in claim 2, wherein the determining the wave period comprises setting a start point of an electrocardiogram signal period for maintaining a state, in which the differential value of the variation value is greater than a first threshold, for at least a period of time, as a start point of the wave period.

5. The method as claimed in claim 4, wherein the detecting comprises setting a point of the electrocardiogram signal with at least one of the maximum size and inclination in the wave period, as the apex.

6. The method as claimed in claim 1, further comprising displaying a waveform of the electrocardiogram signal on a display.

7. The method as claimed in claim 1, wherein the wave period is a QRS complex period.

8. A method of displaying an electrocardiogram signal, the method comprising:
measuring an electrocardiogram signal while irradiating a body portion with an ultrasonic signal and receiving a reflected signal;
configuring an ultrasonic image based on the received signal;
determining an wave period based on an inclination of the measured electrocardiogram signal;
detecting an apex of the electrocardiogram signal with a maximum size in the wave period;
determining a waveform of the electrocardiogram signal based on the wave period and the apex; and
displaying the determined waveform of the electrocardiogram signal together with the configured ultrasonic image.

9. The method as claimed in claim 8, wherein the displaying the determined waveform of the electrocardiogram signal together with the configured ultrasonic image comprises synchronizing and displaying the waveform of the electrocardiogram signal and the ultrasonic image.

10. An electrocardiogram signal detecting apparatus comprising:
an electrocardiogram signal measurer configured to measure an electrocardiogram signal;
a wave period determiner configured to determine a wave period based on an inclination of the measured electrocardiogram signal; and
an apex detector configured to detect an apex of the electrocardiogram with at least one of a maximum size and inclination signal in the wave period.

11. The electrocardiogram signal detecting apparatus as claimed in claim 10, wherein the wave period determiner is configured to determine the wave period using a variation value for each period of the received electrocardiogram signal and a differential value of the variation value.

12. The electrocardiogram signal detecting apparatus as claimed in claim 11, wherein the wave period determiner is configured to determine a maximum threshold and a minimum threshold for calculation of the wave period based on an average value and a maximum value of the differential value of the variation value, detect an apex with the maximum differential value of the variation value among points with the differential value of the variation value, greater than the maximum threshold, and set a start point and an end point of an electrocardiogram signal period for maintaining a state, in which the differential value of the variation value is smaller than the minimum threshold based on the apex, for a period of time, as a start point and an end point of the wave period, respectively.

13. The electrocardiogram signal detecting apparatus as claimed in claim 11, wherein the wave period determiner is configured to set a start point of an electrocardiogram signal period for maintaining a state, in which the differential value of the variation value is greater than a first threshold, for at least a period of time, as a start point of the wave period.

14. The electrocardiogram signal detecting apparatus as claimed in claim 13, wherein the apex detector is configured to set a point with at least one of the maximum size and inclination of the electrocardiogram signal in the wave period, as the apex.

15. The electrocardiogram signal detecting apparatus as claimed in claim 10, further comprising a display configured to display a waveform of the electrocardiogram signal.

16. The electrocardiogram signal detecting apparatus as claimed in claim 10, wherein the wave period is a QRS complex period.
